(19) United States
(12) Patent Application Publication
(10) Pub. No.: US 2013/0122038 A1
(43) Pub. Date: May 16, 2013

(54) HETEROLOGOUS PRIME-BOOST IMMUNIZATION USING MEASLES VIRUS-BASED VACCINES

(71) Applicants: Crucell Holland B.V., CN Leiden (NL); THE UNITED STATES OF AMERICA AS REPRE, Rockville, MD (US)

(72) Inventors: Katarina RADOSEVIC, ML Nootdorp (NL); Mario Roederer, Washington, DC (US); Diane Bolton, Washington, DC (US); Jerôme H.H.V. Custers, AB Alphen aan den Rijn (NL)

(73) Assignees: THE UNITED STATES OF AMERICA AS REPRESENTED BY THE SECRETARY OF THE DEPARTMENT, Rockville, MD (US); Crucell Holland B.V., CN Leiden (NL)

(21) Appl. No.: 13/675,447

(22) Filed: Nov. 13, 2012

Related U.S. Application Data
(60) Provisional application No. 61/559,359, filed on Nov. 14, 2011.

(30) Foreign Application Priority Data
Feb. 1, 2012 (EP) 12153412.7

(51) Int. Cl. A61K 35/76

(52) U.S. Cl. CPC A61K 35/761
USPC 424/199.1

(57) ABSTRACT

The invention provides reagents and methods for heterologous prime-boost immunization regimens. In particular, the invention provides reagents and methods for use in a paramyxovirus-based prime and adenovirus-based boost immunization system, wherein the immunization induces an immune response to a foreign antigen.

### RELATED APPLICATIONS

This application claims priority to U.S. Provisional Application Ser. No. 61/559,359 filed Nov. 14, 2011 and European Patent Application Serial No. EP12153412.7, filed Feb. 1, 2012.

### STATEMENT OF U.S. GOVERNMENT INTEREST

This invention was created in the performance of a Cooperative Research and Development Agreement with the National Institutes of Health, an Agency of the Department of Health and Human Services. The Government of the United States has certain rights in this invention.

A sequence listing txt file, 11-919-PRO.txt, created on Nov. 14, 2011 in the size of 24 kilobytes, submitted herewith is incorporated by reference in its entirety.

### FIELD OF THE INVENTION

The present invention generally relates to reagents and methods for immunization. More particularly, the invention relates to prime-boost immunization, administered either prophylactically or therapeutically, against a foreign antigen or foreign antigen of a pathogen.

### BACKGROUND OF THE INVENTION

Vaccine development has been a major driving force in controlling and eradicating infectious diseases in recent human history. The adaptability and versatility of the body's immune system may be the ultimate source to combat the emergence of drug resistant pathogenic strains.

Live attenuated measles vaccine have been extensively used.

The safety record, immunogenicity and manufacturability make live attenuated measles virus an attractive candidate to develop as a recombinant vaccine vector.

In a recombinant measles vaccine (rMV) vector, an antigen from another pathogen is incorporated into the measles genome (see e.g. WO 97/06270). During the replication and transcription of rMV in a cell, the transgene is expressed together with viral proteins and presented to the host immune system, inducing a transgene-specific immune response. Thus a multivalent vaccine vector would induce not only strong immunity and protection against measles but also against another pathogen. A number of different transgenes, including genes from human papilloma virus, SARS coronavirus, West Nile virus, and human and simian immunodeficiency viruses (HIV/SIV), have been stably incorporated into the recombinant measles genome, with demonstrated transgene protein expression (see e.g., Cantarella et al., Vaccine 2009, 27:3385-90; Despres et al., J Infect Dis 2005, 191:207-14; Liniger et al., Vaccine 2008 26:2164-74; Brandler et al., Vaccine 2010 28:6730-9).

In vivo studies with measles virus and recombinant measles vectors have traditionally been performed in immunocompromised (IFNα receptor −/−) mice transgenic for human CD46 receptor, a measles virus receptor (Cantarella et al., Vaccine 2009, 27:3385-90; Lorin et al., J Virol 2004 78:146-57; Zuniga et al., Vaccine 2007 25:2974-83; Mrkic et al., J Virol 1998 72:7420-7). Immunogenicity studies in these animals demonstrated that recombinant measles vectors induce not only strong immune response against measles but also against the transgene (Liniger et al., Vaccine 2008 26:2164-74; Lorin et al., J Virol 2004 78:146-57; Zuniga et al., Vaccine 2007 25:2974-83; Brandler et al., Vaccine 2010 28:6730-9).

In immunocompetent mice, however, the immune response against the transgene is generally low even though measles-specific responses are well induced, possibly reflecting the inability of measles vector to efficiently replicate in these animals. Initial immunogenicity study performed in non-human primates using rMV vector based on the licensed Edmonston Zagreb (EZ) vaccine strain MORATEN® as the backbone and expressing SIVgag (rMV-Gag) failed to demonstrate transgene specific responses while animals developed measles specific immune responses (Tangy et al., Viral Immunol 2005 18:317-26). The results would argue against the use of recombinant measles vector as a stand-alone vaccine against other pathogens.

Thus, there remains a need in the art for vaccination regimens that have improved efficacy for inducing immune responses compared to recombinant measles virus vectors.

### SUMMARY OF THE INVENTION

The invention thus provides methods and reagents directed towards immunization, prophylactic and/or therapeutic, that are not hampered by the limitations found in the prior art.

Recombinant paramyxovirus, exemplified in the form of recombinant attenuated measles virus (rMV) derived from the Edmonston Zagreb vaccine strain, was engineered to express simian immunodeficiency virus (SIV) Gag protein (SEQ ID NO:1 (DNA), SEQ ID NO:2 (protein)) for the purpose of evaluating the immunogenicity of rMV as a vaccine vector in rhesus macaques. rMV-Gag immunization alone elicited robust measles-specific humoral and cellular responses, but failed to elicit transgene (Gag) specific immune response, following aerosol or combined intratracheal/intramuscular delivery. Thus, the rMV vector may not be suitable as a stand-alone vaccine against all pathogens.

However, when administered as a prime vaccine to a heterologous boost with a different recombinant virus, for example recombinant adenovirus (rAd) expressing the same transgene (rAd5-Gag), the rMV-Gag priming unexpectedly and significantly enhanced Gag-specific T lymphocyte responses following rAd5-Gag immunization. The transgene cellular response priming ability of rMV was highly effective even when using a suboptimal dose of rAd for the boost. These data surprisingly demonstrated the feasibility of using rMV as a priming component of heterologous prime-boost vaccine regimens, e.g., for pathogens for which strong cellular responses are required.

Accordingly, in one aspect, the invention provides heterologous prime-boost immunization methods for inducing an immune response in a mammal to a foreign antigen, comprising the steps of (a) administering to a mammal a priming immunogenic composition comprising a recombinant paramyxovirus; and (b) administering to the mammal a first boosting immunogenic composition comprising a different recombinant virus, wherein the recombinant paramyxovirus and recombinant boosting virus each comprises a transgene encoding an epitope of the foreign antigen. In certain embodiments, the recombinant paramyxovirus and/or the recombinant virus of the boosting immunogenic composition comprise live-attenuated viruses. In certain particular embodiments, the paramyxovirus comprises measles virus or mumps virus. In certain preferred embodiments the paramyxovirus is a measles virus. In certain other particular embodiments, the first boosting virus is a recombinant adenovirus. In certain other particular embodiments, the epitope is from a protein of a bacterium, a virus or a parasite. In certain further embodiments, the epitope is from HIV Gag protein (SEQ ID NO:3 (DNA), SEQ ID NO:4 (protein)). In certain other embodiments, the mammal is a human.

In certain particular embodiments, the inventive methods comprise administering to the mammal more than one (i.e., 2, 3, 4, or more) boosting immunizations. In particular, certain advantageous embodiments of the inventive method further comprise administering to the mammal a second boosting immunogenic composition. In certain particular embodiments, the first boosting immunogenic composition comprises a recombinant measles virus, and the second boosting immunogenic composition comprises a recombinant adenovirus. In certain other particular embodiments, the first and second (or additional) boosting immunogenic compositions comprise a recombinant adenovirus. In certain other embodiments, the priming or boosting immunogenic composition further comprises an immune adjuvant.

The priming immunogenic composition or the boosting immunogenic composition can be administered to the mammal by any known route of administration to one of ordinary skill in the field, including without limitation intratracheal, intramuscular and aerosol routes. In certain particular embodiments, the priming immunogenic composition and the first and (when administered) second boosting immunogenic compositions are administered by the aerosol route. In certain other embodiments, the priming immunogenic composition is administered by the intratracheal route, and the first and (when administered) second boosting immunogenic compositions are administered by the intramuscular route. In yet other embodiments, the priming and first and (when administered) second boosting immunogenic compositions are administered by the aerosol route.

Administering a vaccine to a mammal can elicit humoral and/or cellular immune responses in the mammal. In certain embodiments, the immune response comprises a cellular immune response. In certain particular embodiments, the cellular immune response comprises a T cell-mediated immune response; in certain particular embodiments, the T cell-mediated immune response comprises a CD8+ T cell-mediated immune response.

In a further aspect, the invention provides methods of inducing an immune response in a mammal to a foreign antigen comprising the steps of (a) administering to a mammal a recombinant measles virus-based vaccine in a priming immunization; and (b) administering to the mammal a recombinant adenovirus-based vaccine in a boosting immunization, wherein the recombinant measles virus and the recombinant adenovirus each comprise a transgene that encodes an epitope of the foreign antigen. In certain particular embodiments, the recombinant measles virus-based vaccine is administered at an effective amount to induce an immune response to measles virus.

In yet another aspect, the invention provides kits for use in prime-boost vaccinations comprising a first container comprising a priming composition comprising a recombinant measles virus and a second container comprising a boosting composition comprising a recombinant adenovirus, wherein the recombinant measles virus and the recombinant adenovirus each comprises a transgene that encodes an epitope of a foreign antigen. In certain particular embodiments, the kit further comprises a buffer. In certain other embodiments, the kit further comprises instructions for use.

Each and every embodiment described throughout the application can be combined, and can be applied to each and every aspect of the invention described herein. Further, the methods and reagents of the various aspects of the instant invention can be used prophylactically. Alternatively, they can be used therapeutically.

Specific embodiments of the present invention will become evident from the following more detailed description of certain preferred embodiments and the claims.

### BRIEF DESCRIPTION OF THE FIGURES

FIG. 1 shows the Experimental Schema for the heterologous prime-boost immunization regimens. (A) In Study A, rhesus macaques were immunized with 5×10⁴ pfu/dose (1× dose; group 1) or 10⁶ pfu/dose (20× dose; group 2) of rMV-Gag (recombinant measles virus comprising the SIV Gag transgene) twice by aerosol delivery followed by aerosol immunization with rAd5-Gag (recombinant adenovirus comprising the SIV Gag transgene) at a dose of 10¹⁰ pfu. A third group received a single dose of rAd5 priming immunization followed by two boosting immunizations with the 20×rMV-Gag dose, all by aerosol delivery. Immunizations were administered eight weeks apart. (B) In Study B, animals received either rMV-null (n=6) or rMV-Gag (n=16) administered twice: first by the intratracheal route (IT) at week 0 and second by the intramuscular route (IM) at week 12. Half of the animals received the 1× dose and the other half the 20× dose. All animals received a heterologous boost of 10⁷ PU/dose (particle unit) of rAd5-Gag intramuscularly (IM) 20 weeks after the second rMV immunization.

FIG. 2 presents graphs showing results of the heterologous prime-boost immunization regimens as in Study A. (A) Serum IgG responses to MV were measured by ELISA and presented in optical density units (OD). Pre-immune and week 8 responses are shown for each animal with lines coded according to vaccine group assignment. The two MV-seropositive animals assigned to rMV-Gag prime groups are indicated (+, 1×; *, 20×). (B) BAL MV N-specific CD4⁺ (“Subset 4”) and CD8⁺ (“Subset 8”) T cell responses were measured four weeks after the second immunization by ICS (intracellular cytokine staining) for IFNγ, IL-2, and TNF after in vitro peptide pool stimulation. The total percentage of each subset of T cells that produce any combination of these cytokines is plotted for each animal each depicted by a unique symbol. (C) PBMC CD8⁺ T cell Gag-specific responses were measured by peptide pool stimulation and ICS as in (B) and shown over time for each animal. (D) BAL Gag-specific T cell responses were measured as in (B) and shown over time. (E) BAL Gag-specific T cell responses from (D) are shown for all animals at four weeks after rAd5 immunization.

FIG. 3 presents graphs showing humoral immunogenicity after intratracheal and intramuscular rMV immunizations followed by suboptimal rAd5 boost as in Study B. (A) Serum MV-specific IgG titers against MV lysate are plotted for each animal in Study B grouped by vaccine regimen at the indicated weeks after the first rMV immunization. ELISA IgG measurements are plotted as units/ml. (B) MV 50% neutralization titers are shown for each animal as in (A) where the rMV-null and -Gag data were combined and grouped based on rMV dose. Bar indicates the mean value for each vaccine group. Protective titer of 120 mIU/ml is indicated by a dotted line and asterisk.

FIG. 4 presents graphs showing Gag-specific cellular immunogenicity after intratracheal and intramuscular rMV immunizations followed by suboptimal rAd5 immunization. Rhesus macaques primed with rMV-null, rMV-Gag₁ₓ, or rMV-Gag₂₀ₓ were boosted with 10⁷ PU rAd5 intramuscularly at week 32 as in Study B. (A) Gag-specific PBMC T cell responses are shown as measured by ELISpot; statistical tests represent comparison to week 32. Bars depict the interquartile range for each group; (#) and (+) indicate significant difference from the week 1-time point within each group by Wilcoxon rank-sum and Student's t-test, respectively. (B) PBMC CD4⁺ (left) and CD8⁺ (right) T cell responses to SIV Gag measured by ICS as in FIG. 2C were shown, either before (week 14) and after (week 36) the rAd5 boost. Statistical comparison to week 14 pre-rAd5 values within each group is indicated. (C) Gag-specific BAL T cell responses for the CD4⁺ (left) and CD8⁺ (right) subsets measured by ICS as in (B) are shown at week 36, 4 weeks after the rAd5 boost. Not all animals are plotted due to insufficient BAL T cell subset populations detected.

### DETAILED DESCRIPTION OF THE INVENTION

All publications, patents and published patent applications cited herein are hereby expressly incorporated by reference in their entirety for all purpose.

Methods well known to those skilled in the art can be used to construct expression vectors and recombinant bacterial cells according to this invention. These methods include in vitro recombinant DNA techniques, synthetic techniques, in vivo recombination techniques, and PCR techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

In one aspect, the invention provides heterologous prime-boost immunization methods for inducing an immune response in a mammal to a foreign antigen comprising the steps of (a) administering to a mammal a priming immunogenic composition comprising a recombinant paramyxovirus; and (b) administering to the mammal a first boosting immunogenic composition comprising a different recombinant virus, such as recombinant adenovirus, wherein the recombinant paramyxovirus and recombinant adenovirus each comprises a transgene encoding an epitope of the foreign antigen.

In certain embodiments, the mammal is not immunocompromised, or wild-type with respect to its immune system. It has surprisingly found herein that in immunocompetent mammals the initial vaccination with the recombinant paramyxovirus led to barely detectable immune responses to the foreign antigen. This would discourage the skilled artisan, as usually (for other vectors) significant immune responses are expected after a first vaccination. However, the present inventors persevered and found out that in spite of the results with the first vaccination with recombinant paramyxovirus, nevertheless the boosting by the different recombinant virus such as adenovirus, surprisingly results in a good immune response. This could not be predicted based on the knowledge of the skilled artisan prior to the instant invention.

Paramyxovirus-based vaccines, such as measles virus-based vaccines, used as a standalone vaccine elicited weak or no immunogenicity to a foreign antigen, i.e., an antigen exogenous to the viral vector and the host, in an immune competent host. It was unexpectedly discovered by the instant inventors that, despite its weak immunogenicity as a standalone vaccine, paramyxovirus-based vaccine, when used as a priming vaccine in a heterologous prime-boost immunization regimen, greatly enhanced T cell-mediated immune response to the foreign antigen.

Paramyxovirus is a group of single-stranded negative-sense RNA virus. Viral replication is necessary for expression of a transgene in the viral genome. Recombinant paramyxovirus can be prepared according to well-known methods, e.g. described in WO 97/06270, U.S. Pat. No. 7,993,924, WO 99/63064, WO 01/09309, WO 2004/00876, WO 2004/01051, EP 2110382 and WO 2004/113517, all incorporated by reference herein. The Edmonston Zagreb measles strain used herein as an exemplary vector is a highly attenuated and thus superbly safe strain especially suitable for use in infants against measles virus infection. It was unexpectedly discovered that the attenuated paramyxovirus-based viral vector, by itself insufficient to effectively induce immune response to a transgene, is sufficient to prime CD8+ T-cell response specific for a foreign antigen, such as HIV Gag protein, in a heterologous prime-boost immunization regimen. In view of the similar properties of paramyxoviruses, it is expected that the findings herein with a recombinant measles virus (rMV) will apply to any paramyxovirus. Hence, where the application refers to rMV, this is intended to include any recombinant vectors from the group of paramyxoviruses, such as measles virus, mumps virus, etc.

In certain embodiments of the invention, the heterologous boost vaccine is a viral vector expressing the transgene, including without limitation, a recombinant adenovirus-based viral vector, adeno-associated virus-based viral vector, vesicular stomatitis virus-based viral vector, and pox virus-based viral vector, such as modified vaccinia Ankara (MVA) virus. In certain particular embodiments, the boost vaccine comprises live attenuated virus. In certain other embodiments the boost vaccine comprises a virus which is not within the paramyxovirus family. In certain particular embodiments, the heterologous boost vaccine is a recombinant adenovirus-based viral vector.

As used herein, the term “heterologous prime-boost immunization” refers to an immunization regimen that comprises immunizing a mammal with a priming immunization and at least one boosting immunization, wherein the priming immunization and the at least one boosting immunization comprise different types of vaccine. For example, an immunization regimen consisting of a measles virus-based priming immunization followed by an adenovirus-based boosting immunization constitutes a heterologous prime-boost immunization regimen, whereas an immunization regimen consisting of a measles virus-based priming immunization followed by only one measles virus-based boosting immunization does not. It is understood that the term heterologous prime-boost immunization intends to encompass immunization regimens in which one of the multiple boosting immunizations comprises the same recombinant viral vector as used in the priming immunization and thus a “homologous boost,” either of the same or different doses, as long as at least one of the multiple boosting immunizations comprises a viral vector that is different from that used in the priming immunization.

The term “prime immunization,” “priming immunization,” or “primary immunization” refers to primary antigen stimulation by using a paramyxovirus-based recombinant viral vector according to the instant invention. The mammal that receives the priming immunization may or may not have already been exposed to the vector of the prime immunization, and/or the pathogen against which the prime immunization is designed, for instance by natural infection.

The term “boost immunization,” “boosting immunization,” or “secondary immunization” refers to additional immunization administered to or effective in a mammal after the primary immunization. In various embodiments, the boost immunization is administered at a dose higher than, lower than or equal to the effective dose that is normally administered when the boost immunization is administered alone without priming. In certain advantageous embodiments, the boost immunization is administered to the mammal at a dose lower than the effective dose that would be used when the immunization is administered to the mammal alone without priming.

The terms “vaccine,” “vaccine composition,” and “immunogenic composition” are used interchangeably throughout this application. In certain embodiments of the invention, the vaccine or immunogenic composition comprises a recombinant measles virus or a recombinant adenovirus. The recombinant measles virus or recombinant adenovirus each comprise a transgene expressing or encoding an epitope of a foreign antigen. In certain particular embodiments, the recombinant measles virus and/or recombinant adenovirus are live attenuated viruses that maintain the ability to replicate and transcribe the viral genome inside a cell. For the recombinant adenovirus, the virus is preferably replication-deficient, e.g. by virtue of mutations or deletions in the E1-region. In certain other embodiments, the immunogenic composition further comprises an immune adjuvant.

The prime and boost vaccine compositions may be administered via the same route or they may be administered via different routes. The boost vaccine composition may be administered one or several times at the same or different dosages. It is within the ability of one of ordinary skill in the art to optimize prime-boost combinations, including optimization of the timing and dose of vaccine administration.

An immunogenic composition or vaccine that is “specific for a pathogen,” “against a pathogen” or “to a pathogen” means that the immunogenic composition or vaccine, when administered to a mammal, elicits an immune response specific for the pathogen. An immunogenic composition or vaccine that is “specific for a foreign antigen,” “against a foreign antigen,” or “to a foreign antigen” indicates that the immunogenic composition or vaccine, when administered to a mammal, elicits an immune response specific for the foreign antigen. It is within the ability of one of skilled in the art, and further taught by the instant disclosure, how to discern specific immune response from non-specific immune response.

The prime vaccine composition comprises a recombinant paramyxovirus. In certain particular embodiments, the paramyxovirus comprises measles virus or mumps virus. In most particular embodiments, the paramyxovirus is measles virus. Other suitable paramyxovirus-based viral vector includes without limitation mumps virus-based vector, human parainfluenza virus-based vector, human metapneumovirus-based vector, Newcastle disease virus-based vector, Sendai virus-based vector, and canine distemper virus-based vector. Suitable measles virus-based viral vector includes without limitation the following vaccine strains Edmonston Zagreb, Schwarz, Moraten, Rubeovax, Leningrad 4, AIK-C, Connaught, and CAM-70.

The boost vaccine composition comprises a recombinant adenovirus, also referred to as recombinant adenoviral vectors. The preparation of recombinant adenoviral vectors is well known in the art. Adenoviruses for use as vaccines are well known and can be manufactured according to methods well known to the skilled person. The adenoviruses used for the invention are recombinant adenoviruses and can be of different serotypes, for instant human serotype 5 (Ad5), or 26 (Ad26), or 35 (Ad35). Recombinant adenoviruses can be produced to very high titers using cells that are considered safe, and that can grow in suspension to very high volumes, using medium that does not contain any animal- or human derived components. Also, it is known that recombinant adenoviruses can elicit a strong immune response against the protein encoded by the heterologous nucleic acid sequence in the adenoviral genome. In the genome of the adenovirus, nucleic acid encoding the antigen or an immunogenic portion thereof is operably linked to expression control sequences. In certain embodiments, an adenoviral vector according to the invention is deficient in at least one essential gene function of the E1 region, e.g. the E1a region and/or the E1b region, of the adenoviral genome that is required for viral replication. In certain embodiments, an adenoviral vector according to the invention is deficient in at least part of the non-essential E3 region. In certain other embodiments, the vector is deficient in at least one essential gene function of the E1 region and at least part of the non-essential E3 region. The adenoviral vector can be “multiply deficient,” meaning that the adenoviral vector is deficient in one or more essential gene functions in each of the two or more regions of the adenoviral genome. For example, the aforementioned E1-deficient or E1-, E3-deficient adenoviral vectors can be further deficient in at least one essential gene of the E4 region and/or at least one essential gene of the E2 region (e.g., the E2A region and/or E2B region). As known to the skilled person, in case of deletions of essential regions from the adenovirus genome, the functions encoded by these regions have to be provided in trans, preferably by the producer cell, i.e. when parts or whole of E1, E2 and/or E4 regions are deleted from the adenovirus, these have to be present in the virus producer cell, for instance integrated in the genome, or in the form of so-called helper adenovirus or helper plasmids. In certain embodiments, the adenovirus lacks at least a portion of the E1-region, e.g. E1A and/or E1B coding sequences, and further comprises heterologous nucleic acid encoding the antigen of interest or an immunogenic part thereof. Adenoviral vectors, methods for construction thereof and methods for propagating thereof, are well known in the art and are described in, for example, U.S. Pat. Nos. 5,559,099, 5,837,511, 5,846,782, 5,851,806, 5,994,106, 5,994,128, 5,965,541, 5,981,225, 6,040,174, 6,020,191, and 6,113,913, and Thomas Shenk, “Adenoviridae and their Replication”, M. S. Horwitz, “Adenoviruses”, Chapters 67 and 68, respectively, in Virology, B. N. Fields et al., eds., 3d ed., Raven Press, Ltd., New York (1996), and other references mentioned therein. Methods for producing and purifying adenoviruses are disclosed in for example WO 98/22588, WO 00/32754, WO 04/020971, U.S. Pat. No. 5,837,520, U.S. Pat. No. 6,261,823, WO 2005/080556, WO 2006/108707, WO 2010/060719, and WO 2011/098592, all incorporated by reference herein. One of skill will recognize that elements derived from multiple serotypes can be combined in a single recombinant adenovirus vector. Thus, a chimeric adenovirus that combines desirable properties from different serotypes can be produced.

An adenovirus suitable for use according to the invention can be a human adenovirus of any serotype. It can also be an adenovirus that infects other species, including but not limited to a bovine adenovirus (e.g. bovine adenovirus 3, BAdV3), a canine adenovirus (e.g. CAdV2), a porcine adenovirus (e.g. PAdV3 or 5), or a simian adenovirus (which includes a monkey adenovirus and an ape adenovirus, such as a chimpanzee adenovirus). Non-limiting exemplary serotypes of human adenovirus that can be used according to the invention include Ad2, 5, 11, 26, 34, 35, 36, 48, 49 and 50. Non-limiting exemplary types of chimpanzee adenovirus vectors (see e.g. U.S. Pat. No. 6,083,716; WO 2005/071093; Farina et al, 2001, J Virol 75: 11603-13; Cohen et al, 2002, J Gen Virol 83: 151-55; Kobinger et al, 2006, Virology 346: 394-401; Tatsis et al., 2007, Molecular Therapy 15: 608-17; see also review by Bangari and Mittal, 2006, Vaccine 24: 849-62; and review by Lasaro and Ertl, 2009, Mol Ther 17: 1333-39) that can be used according to the invention include Pan-5 (also referred to as C5, AdC5 or SAdV22), Pan-6 (C6, AdC6 or SAdV23), Pan-7 (C7, AdC7 or SAdV24) and Pan-9 (CV-68, C68, AdC68, or SAdV25), ChAd3 (AdC3), ChAd63 (AdC63) and other chimpanzee adenovirus serotypes disclosed in WO 2005/071093, or in WO 2010/086189, or in WO 2010085984. Further vectors based on ape adenovirus isolates have been described in detail by (Roy et al, 2010, J Gene Med, DOI: 10.1002/jgm.1530), some of which may be based on serotypes that are very similar or the same as certain ones previously reported but not yet with complete details by others. In certain embodiments, an adenovirus according to the invention is thus a simian adenovirus, such as a chimpanzee adenovirus, which include but is not limited to any of the serotypes mentioned above (e.g. the serotypes referred to in table 3 of Roy et al, 2010, J Gene Med, DOI: 10.1002/jgm.1530, incorporated by reference in its entirety herein). The sequences of most of the human and non-human adenoviruses mentioned above are known, and for others can be obtained using routine procedures.

Preparation of recombinant adenovirus vectors, and suitable cell lines for propagation thereof, are well known for both human as well as nonhuman adenoviruses, and can for instance be performed according to the description hereinabove, and/or according to the disclosure in the cited references, which are incorporated by reference in their entirety herein. For example, many (in particular the ones from subgroups C or E) of the chimpanzee adenovirus vectors with deletions in E1 can be propagated in standard (human Ad5-E1 expressing) complementing cells, such as HEK293 or PER.C6 cells (e.g. Roy et al, 2010, supra, e.g. Table 1), but other serotypes may be propagated efficiently on the 293 orf6 cell line that expresses E1 and E4 orf6 from Ad5 (Brough et al, 1996, supra; Nan et al, 2003, supra), or the E4 orf6 of such serotypes may be replaced by an E4 orf6 sequence of a subgroup C or E adenovirus (e.g. human Ad5) for efficient propagation on the readily available and often used HEK293 or PER.C6 or other complementing cells that express E1 from human Ad5 (e.g. WO 03/104467). All references cited are incorporated by reference in their entireties.

The vaccine can be administered to mammals, especially humans, through various routes including without limitation parenteral, intratracheal, intra-arterial, intracutaneous, transdermal, intramuscular, intraperitoneal, intravenous, subcutaneous, aerosol, oral and intranasal administration. In certain embodiments, the prime vaccine is administered by the intratracheal route and the boost vaccine is administered by the intramuscular route. In certain other embodiments, both prime and boost vaccines are administered by aerosol delivery. Other combination of routes can also be used, such as aerosol followed by intratracheal and/or intramuscular, and intratracheal and/or intramuscular followed by aerosol delivery. It is within the knowledge of one skilled in the art, with further instructions provided by the instant disclosure, to select and adjust the route of administration for optimal immunization results.

Intratracheal (IT), intramuscular (IM) and aerosol (AE) deliveries have been used successfully in animal models. IT inoculation ensures delivery of the complete viral dose to the lungs. Attenuated measles Edmonston strain has been shown to replicate in the upper respiratory tract following AE inhalation (de Vries et al., J Virol 2010, 84:4714-24). In addition, IM delivery permits systemic exposure of the antigen and facilitates better peripheral blood cellular response as compared to AE delivery.

As shown below in the example section, enhanced T-cell priming is observed in the non-human primate studies where the priming and boosting immunogenic compositions were delivered by aerosol, IM or IT route. It was unexpectedly discovered that although aerosol delivery of rMV and/or rAd5 resulted in good transgene-specific cellular immune response, IM/IT delivery achieved even better responses. This was surprising because previous studies demonstrated robust humoral immune response by aerosol immunization of recombinant adenovirus or recombinant measles virus comprising a transgene that expresses the HIV Env protein (Lorin et al., supra).

Another aspect of the invention provides methods for dual immunization against measles virus as well as another pathogen. As shown herein, employing rMV as a vaccine vector elicits immune responses against measles virus in all the immunization regimens tested. In certain advantageous embodiments of this aspect, the invention provides methods of dual immunization against measles virus as well as another pathogen such as HIV with a reduced number of immunization events and lower immunization cost.

The term “vaccination” or “immunization” as used herein describes any kind of prophylactic or therapeutic immunization, whether administered after the disease has already been established to improve a clinical situation, or administered for the purpose of preventing a disease or infection from occurring. Therapeutic vaccination can prevent the development of a pathological condition and/or improve a clinical situation. When applied as a preventive agent, it will generally result in a protective immune response.

The term “effective amount” refers to an amount sufficient to elicit an immune response to the intended antigen as a result of the administration of the immunization regimen. The effective amounts for prophylactic and therapeutic vaccination may be the same or may be different. It is within the ability of an ordinarily skilled artisan to determine the effective amount in a given context.

An “epitope” refers to an antigenic determinant of a protein, either truncated or full-length, that is sufficiently antigenic or immunogenic to elicit an immune response. A continuous epitope generally consists of about 5 to about 10 continuous amino acids that form a domain sufficient to elicit a humoral immune response or a T cell-mediated response. A discontinuous epitope, or three-dimensional epitope, can be made up by amino acids located in discontinuous amino acid residues of the protein, which form an antigen determinant recognized by an antibody when the protein is folded in a secondary or tertiary structure. The terms “peptide,” “polypeptide” and “protein” are used interchangeably throughout the application unless specifically indicated otherwise. According to certain particular embodiments of the invention, the priming and boosting immunogenic composition each contains a transgene encoding at least one epitope of a foreign antigen, wherein the epitope is the same in the priming and boosting composition. In certain other embodiments, additional epitopes of the same or different foreign antigens may optionally be encoded by the transgene in either priming or boosting composition, or in both.

The term “foreign antigen” refers to an antigen or protein that is exogenous to the vaccine vector and in certain embodiments is also exogenous to the mammal to be immunized. Similarly, the term “foreign epitope” or “epitope of a foreign antigen” refers to an antigenic or immunogenic epitope that is exogenous to the vaccine vector and in certain embodiments is also exogenous to the mammal to be immunized. In certain embodiments, the recombinant measles virus and/or the recombinant adenovirus comprise a transgene encoding an epitope of a protein that is not an endogenous measles virus protein or an endogenous adenovirus protein. In certain particular embodiments, the recombinant measles virus and/or the recombinant adenovirus comprises a transgene that encodes a fragment of a foreign antigen, particularly a protein from a pathogen, wherein the fragment comprises an antigenic epitope. In certain other embodiments, the recombinant measles virus and/or the recombinant adenovirus comprise a transgene that encodes the full-length protein from a pathogen.

The term “transgene” as used herein refers to a polynucleotide molecule that is exogenous to the vaccine vector and in certain embodiments is also exogenous to the mammal to which the vaccine is administered. In certain non-limiting embodiments, the transgene encodes an antigenic epitope of a protein from Mycobacterium tuberculosis, influenza virus, or HIV. In certain particular embodiments, the transgene encodes an epitope of HIV or SIV Gag protein or HIV or SIV Env protein. In certain other embodiments, the transgene encodes an epitope of a protein from SARS coronavirus, West Nile virus, or any other pathogen, including but not limited to those disclosed herein.

In the context of this invention, the term “pathogen” refers to an entity which through its presence in or on the body leads to or promotes a pathological state which, in principle, is amenable to a preventive or curative immune intervention. The pathogens to which the present invention is applicable includes extracellular bacteria including without limitation Staphylococcus and Streptococcus, Meningococcus and Gonococcus species, species of Neisseria, E. coli, Salmonella, Shigella, Pseudomonas, Diptheria, Bordetella Pertussis, Bacillus pestis, Clostridium species (e.g. Clostridium tetani, Clostridium perfringens, Clostridium novyi, Clostridium septicum); intracellular bacteria including without limitation mycobacteria (e.g. M. tuberculosis) and Listeria monocytogenes; viruses including without limitation retrovirus, hepatitis virus, (human) immunodeficiency virus, herpes viruses, small-pox, influenza, polio viruses, cytomegalovirus, rhinovirus; animal parasites including without limitation protozoa, including without limitation the malaria parasites, helminths, and ectoparasites including without limitation ticks and mites. The pathogens further include Brucella species (e.g. B. melitensis, B. abortus, B. suis, B. canis, B. neotomae, B. ovis), the causative agent for cholera (e.g. Vibrio cholerae), Haemophilus species like H. actinomycetemcomitans, H. pleuropneumoniae, as well as pathogens triggering paratyphoid, plague, rabies, tetanus and rubella diseases. In certain particular embodiments, the methods and reagents of the invention are most useful for preventing or treating HIV infection. Pathogens in this invention are assumed to include, but are not limited to, the eukaryotic cells or their parts that cause various neoplasia, auto-immune diseases and other pathological states of the animal or human body which do not result from microbial infections.

It is within the knowledge of a skilled artisan to identify and determine an antigenic or immunogenic epitope of a protein. Literature, algorithms and software facilitating identification of antigenic epitopes from a primary amino acid sequence are available in the art. For example, it is common knowledge that peptide sequences that are surface-oriented or hydrophilic in nature are generally antigenic regions. See Hopp et al., 1981, Proc. Natl. Acad. Sci. USA, 78:3824-3828; and Harlow et al., Antibodies, a Laboratory Manual, pp. 75-76, 1988, Cold Spring Harbor Laboratory, Cold Spring Harbor, N.Y. It is also known that peptides mapped in the N- and C-termini are usually antigenic peptides because the N-terminus and C-terminus of a protein are often exposed and have a high degree of flexibility. Further, it is known in the art that antigenic regions with high accessibility often border helical or extended secondary structure regions. Algorithms that aid selection of potentially antigenic regions have long been developed and used routinely for antigen design. For example, Hopp et al. and Kyte et al. have developed systems for evaluating the hydrophilic and hydrophobic profile of a polypeptide sequence; and Chou et al. have developed algorithms to identify secondary structure of a polypeptide, such α-helix or β-turn, which aid selection of exposed antigenic regions. See Kyte et al. 1982, J. Mol. Biol., 157:105-132, Chou et al. 1974, Biochemistry, 13:222-245. Many commercial software packages such as MacVector™, DNAStar™, and PC-Gene™ employing similar principles have long been available to one of ordinary skill in the art to analyze and identify hydrophilic and surface accessible antigenic regions of a polypeptide sequence. It is also within the skill in the art to carry out experimentations for testing immunogenicity in an animal.

The transgene comprising the polynucleotide sequence encoding the epitope may be expressed from a separate transcription unit or as a fusion protein or chimeric protein with a protein of the viral vector or with a heterologous protein. In certain embodiments, the epitope may be expressed alone or as part of a fusion protein with a viral protein of the vaccine vector by a transgene present in the genome of a live-attenuated recombinant virus.

The immunogenic compositions or vaccine compositions of the invention can be formulated according to known methods for preparing pharmaceutical compositions, in which the immunogenic substance to be delivered is combined with a pharmaceutically acceptable carrier, diluent or excipient. Suitable carrier, diluent and excipient and the preparation thereof are described, for example, in Genaro, A. O. “Remington: The Science and Practice of Pharmacy.” Lippincott Williams & Wilkins (2005).

For aqueous pharmaceutical compositions used in vivo, sterile pyrogen-free water is preferred. Such formulations will contain an effective amount of the immunogenic substance together with a suitable amount of pharmaceutically acceptable carrier, diluent or excipient in order to prepare pharmaceutically acceptable compositions suitable for administration to a mammal, especially human.

The compositions of the present invention may be in the form of an emulsion, gel, solution, suspension, etc. The vaccine compositions of the present invention can also be lyophilized to produce a vaccine composition in a dried form for ease in transportation and storage. The vaccine compositions of the present invention may be stored in a sealed vial, container, ampule or the like. In the case where the vaccine is in a dried form, the vaccine is dissolved or resuspended (e.g., in sterilized distilled water or a buffer) before administration. An inert carrier such as saline or phosphate buffered saline or any such carrier, in which the vaccine composition has suitable solubility, may be used.

The vaccine compositions of the present invention can optionally be used in concert with an immunoadjuvant and other compounds to support, augment, stimulate, activate, potentiate or modulate the desired immune response of either cellular or humoral type, either prophylactically or therapeutically. Immunoadjuvants include, but are not limited to, various oil formulations such as stearyl tyrosine (ST, see U.S. Pat. No. 4,258,029), the dipeptide MDP, saponin, aluminum hydroxide, aluminum phosphate and lymphatic cytokine Mucosal adjuvants include without limitation cholera toxin B subunit (CTB), a heat labile enterotoxin (LT) from E. coli, and Emulsomes (Pharmos, LTD., Rehovot, Israel). Adjuvants are known in the art to further increase the immune response to an applied antigenic determinant, and pharmaceutical compositions comprising adenovirus and suitable adjuvants are for instance disclosed in WO 2007/110409, incorporated by reference herein. The terms “adjuvant” and “immune stimulant” are used interchangeably herein, and are defined as one or more substances that cause stimulation of the immune system. In this context, an adjuvant is used to enhance an immune response to the antigenic epitope encoded by the transgene of the viral vector.

Suitable adjuvants include an aluminium salt such as aluminium hydroxide gel (alum) or aluminium phosphate, but may also be a salt of calcium, iron or zinc, or may be an insoluble suspension of acylated tyrosine, or acylated sugars, cationically or anionically derivatised polysaccharides, polyphosphazenes, or montanide liposomes. The adjuvant composition may be selected to induce a preferential Th1 response. Moreover, other responses, including other humoral responses, may also be induced. For example, Th1-type immunostimulants which may be formulated to produce adjuvants suitable for use in the present invention may include Monophosphoryl lipid A, in particular 3-de-O-acylated monophosphoryl lipid A (3D-MPL). 3D-MPL is a well-known adjuvant manufactured by Ribi Immunochem, Montana. Other purified and synthetic lipopolysaccharides have been described (U.S. Pat. No. 6,005,099, EP 0729473 B1, EP 0549074 B1). In one embodiment, 3D-MPL is in the form of a particulate formulation having a small particle size less than 0.2 μm in diameter, and its method of manufacture is disclosed in EP 0689454. Saponins are another example of Th1 immunostimulants that may be used. Saponins are well known adjuvants. For example, Quil A (derived from the bark of the South American tree Quillaja Saponaria Molina), and fractions thereof, are described in U.S. Pat. No. 5,057,540, and EP 0362279 B1. The haemolytic saponins QS21 and QS17 (HPLC purified fractions of Quil A) have been described as potent systemic adjuvants, and the method of their production is disclosed in U.S. Pat. No. 5,057,540 and EP 0362279 B1. Particulate adjuvant systems comprising fractions of QuilA, such as QS21 and QS7 are described in WO 96/33739 and WO 96/11711. Yet another example of an immunostimulant is an immunostimulatory oligonucleotide containing unmethylated CpG dinucleotides (“CpG”). CpG is known in the art as being an adjuvant when administered by both systemic and mucosal routes (WO 96/02555, EP 0468520). Such immunostimulants as described above may be formulated together with carriers, such as, for example, liposomes, oil in water emulsions, and or metallic salts, including aluminium salts (such as aluminium hydroxide). For example, 3D-MPL may be formulated with aluminium hydroxide (EP 0689454) or oil in water emulsions (WO 95/17210); QS21 may be advantageously formulated with cholesterol containing liposomes (WO 96/33739), oil in water emulsions (WO 95/17210) or alum (WO 98/15287); CpG may be formulated with alum or with other cationic carriers. Combinations of immunostimulants may also be used, such as a combination of a monophosphoryl lipid A and a saponin derivative (WO 94/00153; WO 95/17210; WO 96/33739; WO 98/56414; WO 98/05355; WO 99/12565; WO 99/11241) or a combination of QS21 and 3D-MPL as disclosed in WO 94/00153. Alternatively, a combination of CpG plus a saponin such as QS21 may also be used.

It is also possible to use vector-encoded adjuvant, e.g. by using heterologous nucleic acid that encodes a fusion of the oligomerization domain of C4-binding protein (C4 bp) to the antigen of interest (e.g. Solabomi et al, 2008, Infect Immun 76: 3817-23). In certain particular embodiments, the adjuvant comprises viral vector encoded adjuvants, including without limitation exogenously expressed cytokines, lymphokines and co-stimulatory molecules encoded by the recombinant viral vectors. For example, the viral encoded adjuvant can be a growth and maturation factor for CTL, such as IL2 or IL-15.

It is within the ability of a skilled artisan to determine whether inclusion of an immune adjuvant is beneficial for eliciting an immune response to the epitope encoded by the transgene in the viral vector. In certain embodiments, the vaccines used in the invention do not comprise further adjuvants.

The term “immunologically effective” or “effective” dosage or amount of the vaccine or immunogenic composition used in this invention means the amount of a single or multiple administrations that is effective for the goal of prevention or treatment. The specific dosage depends on health and body condition of an individual, classified groups (for example: human, nonhuman primates, rodents, etc.), the condition of immune system, the formulations of vaccine, the decision of a health care professional in charge, and other relating factors. Generally, the dosage of the rMV vaccine of the invention ranges from 5×10⁴ to 1×10⁶ pfu per administration. In certain embodiments, the dosage ranges from 1×10⁵ to 1×10⁶ pfu per administration. In certain particular embodiments, the dosage of the recombinant measles virus is 10⁶ pfu per dose. In certain other particular embodiments, the dosage for the recombinant adenovirus is 10¹⁰ pfu per dose. In the methods of the invention, the total dose of the adenovirus provided to a subject during one administration can be varied as is known to the skilled practitioner, and is generally between 1×10⁷ viral particles (vp) and 1×10¹² vp per dose, preferably between 1×10⁸ vp and 1×10¹¹ vp per dose, between 3×10⁸ and 5×10¹⁰ vp, more specifically between 10⁹ and 3×10¹⁰ vp per dose. In certain embodiments, the effective amount to be used in a mammal for each immunization is a suboptimal dosage amount as compared to the dosage amount normally administered to the mammal in a single immunization regimen. The regulation of dosage amounts according to the mentioned method or other standard way for the maximum effect is also regarded as within the knowledge of the people in the field and is further described in the instant disclosure.

The Examples, which follow, are illustrative of specific embodiments of the invention, and various uses thereof. They are set forth for explanatory purposes only, and are not to be taken as limiting the invention.

### EXAMPLES

### Materials and Methods

### Plasmids and Viruses

To create rMV vectors expressing foreign proteins, cDNA corresponding to the antigenome of Edmonston Zagreb vaccine strain was cloned with additional transcription units (ATU) to insert exogenous genes encoding foreign antigens into the viral genome (Zuniga et al. Vaccine 2007, 25:2974-83). Additional nucleotides were added if necessary to comply with the “rule of six,” which stipulates that the number of nucleotides of MV genome must be a multiple of six (Calin et al., 1993, RNA J. Virol. 67:4822-30). In this study 2 measles vectors were used, rMV empty (rMVEZ-null, or rMV-null) and rMV containing SIVgag gene at position 2, between the measles virus P and M genes (rMVEZb2.51Vgag, or rMV-Gag). Viruses were rescued as previously described (Radecke et al., EMBO J 1995, 14(23):5773-84). Vaccine batches were prepared on MRC5 cells (ATCC, Manassas, Va.) as described with few modifications (Liniger et al., Vaccine 2009, 27:3299-305). In brief, recombinant measles vectors were grown in MRC-5 cells in roller bottles at 35° C./5% CO₂ and viruses were harvested at several time points post infection. Viral titers were determined by standard plaque assay on Vero cells (ATCC, Manassas, Va.). Presence of the transgene was confirmed by RT-PCR and sequencing and protein expression was confirmed by western blotting (anti-SIV Gag p27 antibody 2F12, Catalogue #1610, NIH AIDS Research & Reference Reagent Program). Recombinant E1/E3/E4-deleted rAd5 construct expressing GagPol_{SIV} and virus stocks (rAd5-Gag) were generated as previously described (Brough et al., J Virol 1996, 70:6497-501, Gall et al., Mol Biotechnol 2007, 35:263-73).

### Experimental Schema

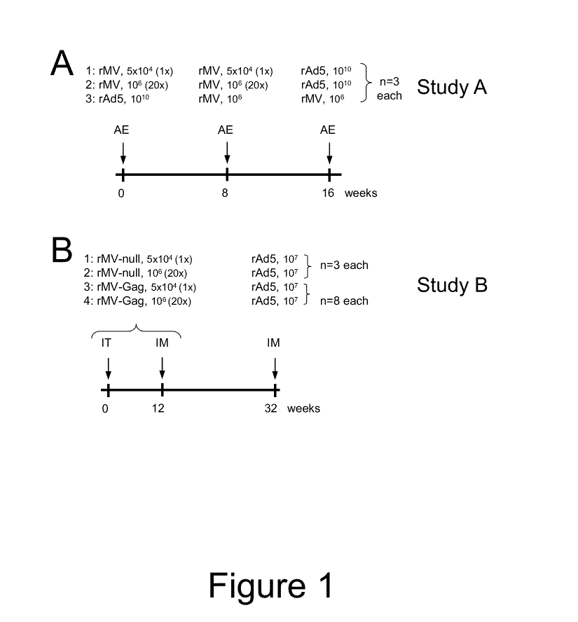

In vitro replication of rMV encoding SIV gag was similar to the parent EZ vaccine strain. Expression of transgene from rMV-Gag was confirmed by western blotting. Immunogenicity to Gag was observed in immunocompetent CD46-transgenic mice but not rhesus macaques (data not shown). To determine the immunogenicity of rMV as a heterologous prime-boost vaccine vector component, rhesus macaques were immunized with rMV-Gag as either a prime or boost to rAd5-Gag using a highly immunogenic AE delivery platform (FIG. 1A, Study A) (Song et al., 2010 Proc Natl Acad Sci USA 107:22213-8). Three immunization regimens were compared: (1) a dose of 5×10⁴ (a single human dose, 1×) pfu of rMV-Gag were administered twice prior to a single 10¹⁰ PU rAd5 AE boosting immunization; (2) a dose of 1×10⁶ pfu of rMV-Gag (20 times a human dose, 20×) were administered twice prior to a single 10¹⁰ PU rAd5-Gag AE boosting immunization; and (3) the 20× dose of rMV-Gag were administered twice as boosting immunizations following a 10¹⁰ PU rAd5-Gag priming (FIG. 1A Study A). All immunizations were spaced 8 weeks apart by aerosol delivery.

In a separate experiment, to further study the ability of rMV to prime for a systemic rAd5 boost, a mucosal IT priming was followed by a first boosting of rMV by IM delivery and a second boosting of a suboptimal dose (10⁷ PU) of rAd5-Gag by IM delivery. The same two doses of rMV (5×10⁴ and 1×10⁶ pfu) used in Study A were also compared in Study B, in which animals received either rMV-Gag or rMV without insert (rMV-null) (FIG. 1B, Study B).

### Animals and Immunizations

Colony-bred Indian-origin rhesus macaques were immunized as described below. In Study A, five animals were seropositive for measles virus at study start. Two of the five seropositive animals were each assigned each rMV-Gag prime group (1× and 20×, n=3 each) and the remaining three were assigned into the rAd5 prime group (n=3). Aerosol immunizations were delivered in 1.0 ml by the e-Flow® Nebulizer System (PARI Pharma, Germany) (Song et al., 2010, Proc Natl Acad Sci USA 107(51):22213-8). In Study B, standard IT and IM immunizations were conducted using 1.0 ml of vaccine. The rMV-null groups (1× and 20×, n=3 each) and rMV-Gag groups (1× and 20×, n=8 each) used in Study B were all measles naïve. Study A animals were housed at Bioqual, Inc. (Rockville, Md.); Study B at New England Primate Research Center of Harvard Medical School (Southborough, M). All animals were maintained in accordance with National Institutes of Health and Harvard Medical School guidelines.

### Measles Virus Antibody Responses

Enzyme immunoassays were used to measure MV-specific IgG in Study A as previously described with some modifications (Lin et al., 2011, Proc Natl Acad Sci USA 108:2987-92). Briefly, sera were diluted 1:100 and incubated overnight at 4° C. with MV-infected Vero cell lysate (1.1 μg/well; Advanced Biotechnologies) coating a Maxisorp 96-well plate (Nalge Nunc International). Plates were washed 4 times with PBS containing 0.05% Tween-20 (PBST). Alkaline phosphatase-conjugated rabbit anti-monkey IgG (BIOMAKOR; Accurate Chemicals) was added to each well (1:1500, 100 ul/well) and plates were incubated for 2 hours at 37° C. followed by four washes in PBST. Plates were developed using the substrate para-nitrophenyl phosphate (SIGMAFAST, Sigma) and the absorbance was read at 405 nm (SoftMax Pro Software v 3.1.1, Molecular Devices) with the average of the three samples reported as optical density. Three negative controls using plasma from naïve monkeys were included in this assay (negative if average optical density <=0.565).

In Study B, anti-MV IgG antibodies were measured using Fisherbrand high protein-binding microtiter plates coated for 5 hrs at room temperature with 60 ng/well of beta-propiolactone-inactivated measles virus (Edmonston strain ATCC VR-24; Virion-Serion, Wurzburg, Germany) in 0.05M carbonate buffer, pH 9.4. Plates were washed with PBS containing 0.05% Tween-20 (PBST), then blocked for 30 min with 2% goat serum (GS) in PBST. Pooled serum from 3 MV-immunized macaques was arbitrarily assigned 1000 units/ml of anti-MV IgG antibody and used as a standard. Individual serum samples were tested at eight serial 3-fold dilutions using a starting dilution of 1/100. After overnight reaction at 4° C., the plates were washed with PBST and treated with 20 ng/well biotinylated goat anti-human IgG (SouthernBiotech, Birmingham, Ala.) for 1 h at 37° C. Plates were washed, then reacted for 30 min at room temperature with 50 ng/well of streptavidin-peroxidase (Sigma). Final development was with tetramethylbenzidine (SouthernBiotech) for 30 min and the reaction was stopped using 2N sulfuric acid stop solution. Absorbance was recorded at 450 nm in a SpectraMax M5 plate reader (Molecular Devices, Sunnyvale, Calif.). Serum was considered positive for anti-MV IgG antibody if the postimmune level was 3.4-fold greater than the preimmune level.

Plaque reduction neutralization assay was used to measure measles virus-specific neutralizing antibody titer following immunization (Albrecht et al., J Virol Methods 1981, 3:251-260). Dilutions of the monkey serum samples were twofold serially diluted from 1:4 to 1:16,384 and standardized to the 3^{rd} WHO international anti-measles standard serum (NIBSC code 97/648, NIBSC, Potters Bar Hertfodshire EN6, 3QG, UK) at 1.0 IU/ml which was twofold serially diluted from 1:8 to 1:256. 200 μl of each serum dilution was incubated with 200 μl of a fixed amount of rMVb2EZ (approximately 70 pfu/ml) for 1 hr at 37° C. No serum was added to the negative controls (virus only). Vero cells seeded in 6-well plates were infected with 200 μl of the serum-MV mix in a humid chamber for 1 hr at room temperature. Then a semi-solid overlay medium (MEM and 1.2% Methocel) was added to every well and the plates were incubated for 6 days at 35° C. and 5% CO₂. After cell fixation and staining (7.4% formaldehyde and 0.4 g crystal violet in 1 liter of PBS pH 7.4), the plaques were counted. The 50% neutralizing end-point titers of each sample in each assay were calculated using the Spearman and Kärber formula. 100% neutralization was defined as no plaques, and 0% neutralization was defined as the geometric mean plaque count of the negative control (virus only). The normalized titer for each sample was obtained by taking the ratio of the estimated 50% neutralizing end-point for the serum sample and the 3^{rd} WHO international standard and multiplying by the WHO titer (1 IU/ml). Normalized titer=(10̂(Log sample−log WHO))*1 IU/ml. Samples with a 50% neutralizing end-point less than 4 (i.e., ≦first dilution on the plate) were considered to be negative; the allocated normalized titer for these samples=(10̂(log(4)−log WHO))*1 IU/ml (Haralambieva et al., Clin Vaccine Immunol 2008, 15:1054-1059). Any titer ≧120 mIU/ml is considered to be protective (Chen et al., J Infect Dis 1990, 162:1036-1042).

### SIV Gag Antibody Measurements

Pre-immune and post-immunization serum and the BAL (bronchoalveolar lavage) and rectal sponge elution were analyzed for humoral responses by ELISA as previously described (Letvin et al., J Virol 2007, 81:12368-74). Rectal secretions were sampled by a modified wick method using Weck-Cel Spears (Windsor Biomedical, Newton, N.H.) as previously described (Kozlowski et al., J Acquir Immune Defic Syndr 2000, 24:297-309). SIV Gag-specific IgA and IgG antibodies were measured using microtiter plates coated with SIV_{mac251} viral lysate lacking detectable envelope protein at 125 ng total protein/well (Advanced Biotechnologies Inc, Columbia, Md.). Total IgA or IgG was measured using plates coated with goat anti-monkey IgA (Rockland, Gilbertsville, Pa.) or IgG (MP BioMedicals, Solon, Ohio). Pooled macaque serum containing previously calibrated amounts of the relevant antibody or immunoglobulin was used for the standards. Secondary reagents were biotinylated goat anti-monkey IgA (25 ng/ml, OpenBiosystems, Huntsville, Ala.) or anti-human IgG (200 ng/ml, Southern Biotech, Birmingham, Ala.) and avidin-labeled peroxidase (0.5 μg/ml, Sigma, St. Louis, Mo.). Plates for antigen-specific ELISAs were developed with tetramethylbenzidine substrate (Sigma) for 30 min, the reaction stopped with 2N sulfuric acid, and read at 450 nm in a SpectraMax M5 plate reader (Molecular Devices, Sunnyvale, Calif.). Total IgA or IgG ELISA absorbance values were recorded at 414 nm after treatment with 2,2-azinobis(3-ethylbenzthiazolinesulfonic acid). The concentration of antigen-specific IgA or IgG was divided by the concentration of total IgA or IgG for each sample to obtain specific activity. Samples were considered to contain significant antibody if the specific activity was ≧mean+3 standard deviations of negative controls and 3.4-fold above the pre-immune specific activity.

### Adenovirus Neutralization Assay

Neutralizing antibodies against Adenovirus serotype 5 were detected by luciferase transgene expression inhibition assay as previously described (Sprangers, et al., J Clin Microbiol 2003, 41:5046-52). Briefly, heat inactivated serum samples were twofold serially diluted in medium (Dulbecco's modified Eagle's medium containing 10% FBS) in duplicate. Serum dilutions ranged from 1/16 to 1/32,768 in an end volume of 50 μl of medium in a 96-well plate. 50 μl of a fixed amount of Adenovirus (Ad5 Adapt Luc, 1×10⁸ PU/ml) was added to each serum dilution and incubated for 30 min at room temperature. Afterwards, 10⁴ A549 cells in 100 μl were added to every well and plates were incubated for 24 hr at 37° C. in 10% CO₂. The medium was discarded, 50 μl of phosphate-buffered saline (PBS) was added and one freeze-thaw cycle performed. Next, 50 μl of Steady-Lite luciferase assay system reagent (Perkin Elmer, Waltham, Mass.) was added to every well and incubated for 15 min at room temperature. An aliquot of 50 μl from each well was transferred to a black and white isoplate and luminescence counts were measured on a 1450 MicroBeta Trilux (Perkin Elmer). Reactions with no serum added, which resulted in maximum luciferase activity, were used as a negative control. The minimum luciferase activity was obtained from wells where no virus was added. As positive control, serum from immunized mice was used. The 90% neutralizing titers were calculated by a non-linear curve fit through the sample data using the minimum luciferase activity control (no virus) as baseline (0%), and the maximum luciferase activity control (no serum) as plateau (100%).

### T Cell Intracellular Cytokine Staining (ICS)

Peripheral blood and BAL were collected from animals following immunization. Single cell suspensions were stimulated with overlapping peptide pools of MV N-protein or SIV Gag at 2.0 μg/ml for 16 hours. Following stimulation, cells were labeled with cell surface markers (CD4-Alexa700APC and CD8-QDot655; unconjugated monoclonal antibodies from Becton Dickenson; conjugations performed in house) and ViViD (to discriminate live/dead cells, LIVE/DEAD, Invitrogen), then fixed and permeabilized (BD Cytofix/Cytoperm, Becton Dickenson) for intracellular cytokine staining with anti-IFNγ-FITC antibody, anti-TNFα-Cy7PE antibody, anti-IL-2-PE antibody, and anti-CD3-Cy7APC antibody (Becton Dickenson). Background from co-stimulation alone (quantified by measuring the staining by anti-CD28 and anti-CD49d antibodies) was subtracted to determine antigen-specific responses.

### T Cell ELISpot Measurements

Multiscreen ninety-six well plates were coated overnight with 100 μl per well of 5 μg/ml anti-human interferon-γ (IFN-γ) (B27; BD Pharmingen, San Diego, Calif.) in endotoxin-free Dulbecco's-PBS (D-PBS). The plates were then washed three times with D-PBS containing 0.1% Tween-20, blocked for 1-4 h with RPMI containing 10% FBS to remove the Tween-20, and incubated with peptide pools at 1 μg/ml and 2×10⁵ PBMCs in triplicate in a 100 μl reaction volume. Following an 18 h incubation at 37° C., the plates were washed nine times with D-PBS containing 0.1% Tween-20 and once with distilled water. The plates were then incubated with 2 μg/ml biotinylated rabbit anti-human IFN-γ (Biosource, Invitrogen, Carlsbad, Calif.) for 2 h at room temperature, washed six times with D-PBS containing 0.1% Tween-20 and incubated for 2.5 h with a 1:500 dilution of streptavidin-AP (Southern Biotechnology, Birmingham, Ala.). After five washes with D-PBS containing 0.1% Tween-20 and one with D-PBS, the plates were developed with NBT/BCIP chromogen (Pierce, Rockford, Ill.), reaction stopped by washing with tap water, air dried, and read with an ELISpot reader using Immunospot software (version 5.0) (Cellular Technology Ltd., Shaker Heights, Ohio).

### Results

### Immunogenicity of Aerosolized rMV

To assess the immunogenicity of rMV vector alone and as a prime or boost vaccine, an aerosol vaccine delivery system was employed (Study A). T cell responses to rAd5-encoded immunogens ranged from 10-60% of CD4 and CD8 subsets in the BAL (bronchoalveolar lavage) following aerosol vaccination was previously observed (Song et al., 2010, Proc Natl Acad Sci USA 107(51):22213-8). Five of the nine animals in Study A were MV seropositive at the start (presumably due to natural MV exposure). These animals were divided into the rAd5 prime group (n=3) and one into each of the 1× and 20×rMV prime groups.

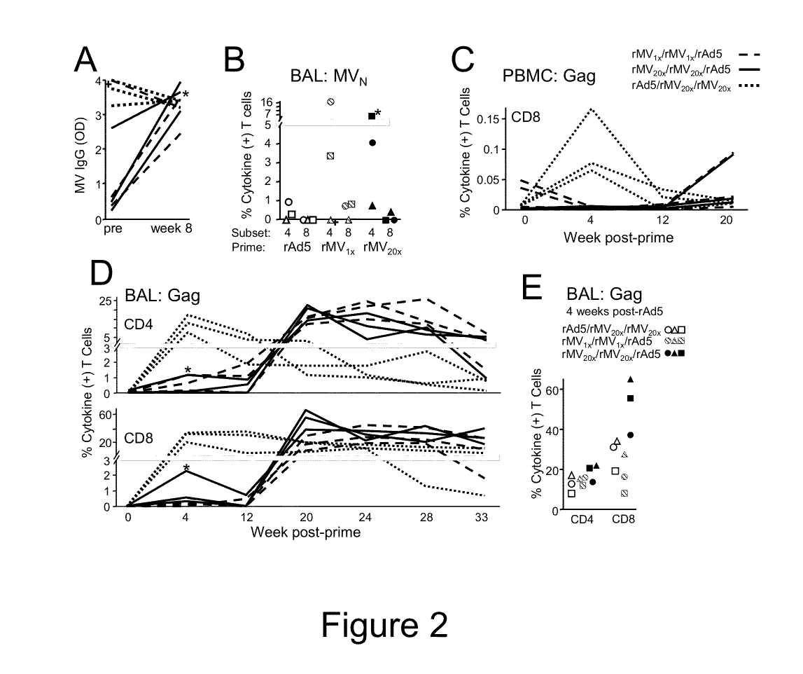

Following aerosol rMV-Gag immunization, all naïve animals mounted significant serum IgG responses to the rMV vector, regardless of the rMV dose (FIG. 2A). The vaccine-induced responses were similar in magnitude to the IgG levels in the five animals that were seropositive at the study start, indicating a robust humoral response to the vaccine. In addition, MV N protein-specific CD4⁺ T cells were also detected in the BAL of all the animals (except for one of the MV seropositive animals) four weeks after the homologous rMV boost, ranging from 1-16% of the CD4⁺ subset, as measured by intracellular cytokine staining (ICS) following ex vivo peptide stimulation (FIG. 2B). There was no significant difference between the 1× and 20×rMV-Gag groups with respect to the magnitude of the T cell response. Moreover, one of the animals with preexisting MV titers had a vigorous N-specific CD4⁺ T cell response (7%), presumably elicited by the rMV-Gag vaccine since MV-seropositive animals that did not receive rMV-Gag (rAd5-Gag prime group) were all <1%. These data are consistent with previous observations that aerosolized vaccine vectors are resistant to neutralization by preexisting serum antibodies to the vector (Song et al., 2010, Proc Natl Acad Sci USA 107(51):22213-8). Low-frequency CD8⁺ T cell responses (<1%) were also observed for half of the animals. Thus, aerosol delivery of rMV-Gag alone elicited robust systemic IgG and local mucosal T cell responses to the MV vector.

To assess the immune response to the SIV Gag immunogen, serum IgG and both blood and BAL T cell responses were measured. Significant Gag-specific IgG was not observed at any time point during the study, including after rAd5-Gag immunization (data not shown). This was surprising since a single aerosol immunization of rAd5 encoding an HIV Env transgene at the same dose was previously shown to elicit serum IgG responses (Song et al., supra). The result may be due to greater immunogenicity of Env relative to Gag. Gag-specific blood T cell responses to the rMV-Gag prime or boost were also undetectable (FIG. 2C and data not shown). By contrast, rAd5-Gag induced demonstrable but not statistically significant Gag-specific CD8⁺ T cell responses 4 weeks post-prime (FIG. 2C, p=0.09). The effects were transient and therefore consistent with no boosting by 20×rMV (Song et al., supra).

Since aerosol immunization typically results in exceptional T cell responses in BAL, and less prominently in peripheral blood T cell responses, BAL was used as the most sensitive site for detecting responses resulting from aerosol immunization. Gag-specific responses by ICS at weeks 4 and 12 following each of the rMV priming immunizations at week 0 and week 8 were measured. Four of six animals primed with rMV had small but notable T cell responses in the BAL 4 weeks after the first prime: two from each of the 1× and 20× groups (FIG. 2D). These responses ranged from 0.6-2.3% of the CD4⁺ or CD8⁺ T cell subsets and declined after the second rMV immunization for all but one animal. By contrast, the rAd5-Gag prime elicited robust and durable Gag-specific BAL T cell responses in all animals: peaking at 8-17% of CD4⁺ and 20-35% of CD8⁺ T cells four weeks after immunization (FIG. 2E). There was no evidence that rMV administered 8 and 16 weeks after rAd5 immunization boosted these responses; however, without matched rAd5 only controls, the possibility of an rMV boost and/or enhanced durability following rAd5 immunization cannot be excluded. A trend was observed in which 20×rMV priming immunization resulted in a greater magnitude CD8⁺ T cell response when measured 4 weeks following rAd5 boosting immunization, as compared to the CD8⁺ T cell response observed in unprimed rAd5 group (FIGS. 2D and 2E, p=0.07). Thus, the results showed that aerosolized rMV-Gag alone was a weak vector platform, but it enhanced responses elicited by a subsequent rAd5-Gag boosting immunization. Gag-specific antibody response, however, was not effectively induced after rAd5 immunization in Study A (data not shown).

Humoral Immunogenicity of Suboptimal rAd5 Dose Primed by rMV

To further investigate the ability of rMV to prime an rAd5 immunization seen in Study A, a second group of rhesus macaques were immunized twice with rMV followed by a low dose of rAd5 that ordinarily would not elicit robust responses (Study B, FIG. 1B). Using the same doses as in Study A, rMV was administered IT and then IM, to assess the effects of rMV mucosal priming followed by a homologous systemic boost. The IM rAd5 boost was given 20 weeks after the second rMV (IM) immunization (week 32).

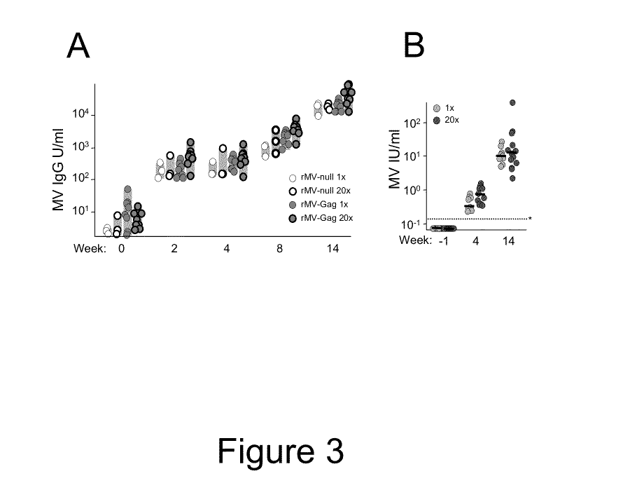

Serum IgG to the measles virus vector was elicited within two weeks of IT delivery in Study B (FIG. 3A), indicating successful vaccine take. Responses were similar for both the null and SIV gag-encoding rMV, ranging from 10²-10³ U/ml at both week two and week four, irrespective of dose. Titers increased slightly by week eight and peaked two weeks after the IM rMV boost (week 14). These titers were sufficient to mediate MV neutralization (FIG. 3B).

By contrast, most animals did not mount a systemic IgG response to the Gag transgene following rMV immunization, as measured by ELISA (data not shown). Gag-specific B cells were likely elicited by rMV in two animals, one in each of the 1× (396) and 20× (339) groups, as these were the only animals that responded to the rAd5 boost. IgG titers in animal 339 increased 6.2-fold from pre-immune levels at week 14, and then 6.3-fold from week 32 to week 34. Animal 396 underwent a 10.3-fold increase in titer from week 0 to 34, with undetectable responses to rMV alone. Mucosal IgA responses were largely undetectable, with no significant Gag-specific responses in the bronchoalveolar lavage (BAL), saliva, or rectum (data not shown). Thus, rMV on its own, without rAd5 boost, elicited strong measles specific immunity but failed to induce transgene specific responses. Together, these data demonstrated robust immunogenicity elicited by rMV-Gag to the measles virus vector but only weak humoral responses to the SIV Gag insert, as observed when the animals were immunized by the AE route.

Cellular Immunogenicity of Suboptimal rAd5 Dose Primed by rMV

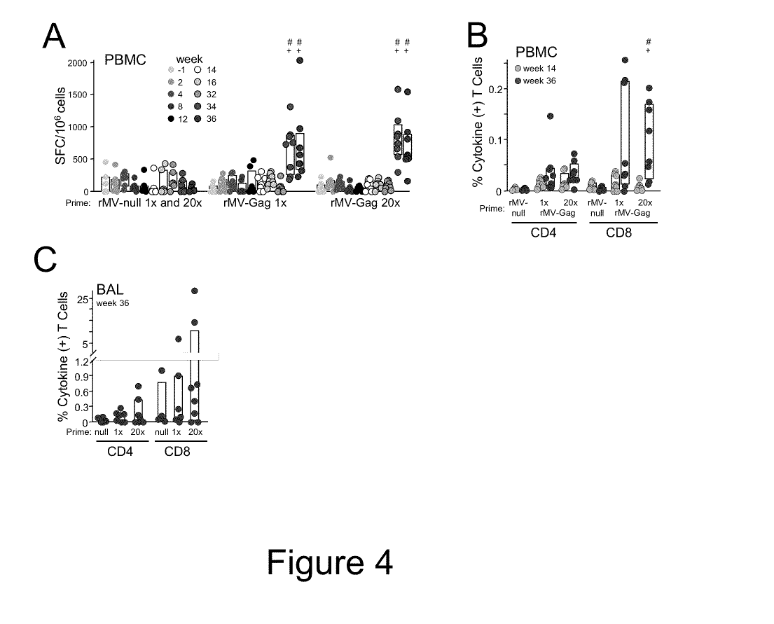

To determine if rMV effectively primed cellular immune responses to rAd5 boost as suggested in Study A, T cell responses following each rMV immunization and the suboptimal dose of rAd5 boost were examined by ICS and ELISpot. The IT rMV immunization failed to induce robust T cell responses in peripheral blood at any time point up to week 12, with no significant difference between pre- and post-immunization levels in either the 1× or 20× vaccine group (FIG. 4A and data not shown). Low-level responses were observed for some animals within each group by ELISpot, but these responses generally did not exceed 250 antigen-specific cells per million cells. Notably, two of the animals (339 and 399) that mounted a significant serum Gag-specific IgG response following rMV immunization also developed modest but durable T cell responses (data not shown). Similarly, Gag ELISpot responses after the second rMV immunization administered intramuscularly were low, with evidence of a boost only seen in the 20× group at week 16 (p=0.02, relative to week 12, FIG. 4A). Following the rAd5 boost, however, significant PBMC T cell responses were detected in both the 1× and 20×rMV-Gag primed groups at two (week 34) and four (week 36) weeks after the rAd5 boost (i.e., p=0.002 and p=0.0008, respectively, versus week 32, FIG. 4A). By contrast, T cell responses to this dose of rAd5 were undetectable without priming (i.e., MV-null).

ELISpot PBMC responses were corroborated by ICS on individual T cell subsets. The results demonstrated that 20×rMV-Gag immunizations primed CD8⁺ T cell responses for the subsequent boost by rAd5-Gag immunization (p=0.01 relative to pre-rAd5, FIG. 4B). The 1×rMV-Gag priming immunization also significantly elevated CD8⁺ T cell responses in half of the animals (p=0.06). By contrast, PBMC CD4⁺ T cell responses were not significantly primed by rMV, as measured by ICS. Large Gag-specific T cell responses, particularly CD8⁺ T cell responses, were also detected in BAL following the rAd5 boost for some animals primed with rMV-Gag (FIG. 4C). Thus, as a component of the heterologous prime-boost, rMV can prime strong cellular responses against the transgene even with a suboptimal boosting vaccine. Combined, these data demonstrated a distinct ability of rMV to prime T cell responses. When delivered either by aerosol or a combination of intratracheal and intramuscular routes of immunization, rMV-Gag increased the magnitude of the gag specific peripheral blood and BAL T-cell responses following the rAd5-Gag boost, particularly among CD8⁺ T cells.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. A heterologous prime-boost immunization method for inducing an immune response in a mammal to a foreign antigen comprising the steps of:
a) administering to a mammal a priming immunogenic composition comprising a recombinant paramyxovirus; and
b) administering to the mammal a first boosting immunogenic composition comprising a recombinant adenovirus,
wherein the recombinant paramyxovirus and recombinant adenovirus each comprises a transgene encoding an epitope of the foreign antigen.

2. The method of claim 1, wherein the recombinant paramyxovirus is a recombinant measles virus.

3. The method of claim 1, wherein the epitope is from a protein of a bacterium, a virus or a parasite.

4. The method of claim 3, wherein the epitope is from human immunodeficiency (HIV) Gag protein.

5. The method of claim 1, wherein the priming immunogenic composition and/or the boosting immunogenic composition are administered to the mammal by intratracheal, intramuscular or aerosol route.

6. The method of claim 1, comprising further administering to the mammal a second boosting immunogenic composition.

7. The method of claim 1, wherein the immune response comprises a T cell immune response.

8. The method of claim 7, wherein the T cell immune response comprises a CD8+ T cell response.

9. The method of claim 1, wherein the mammal is a human.

10. The method of claim 1, wherein the priming and/or boosting immunogenic composition further comprise an immune adjuvant.

11. A method of inducing an immune response in a mammal to a foreign antigen comprising the steps of:
a) administering to a mammal a recombinant measles virus-based vaccine in a priming immunization; and
b) administering to the mammal a recombinant adenovirus-based vaccine in a boosting immunization,
wherein the recombinant measles virus and the recombinant adenovirus each comprise a transgene that encodes an epitope of the foreign antigen.

12. The method of claim 11, wherein the epitope is from a protein of a bacterium, a virus or a parasite.

13. The method of claim 11, wherein the recombinant measles virus-based vaccine is administered in an effective amount to induce an immune response to measles virus.

14. A kit for use in prime-boost vaccination comprising
a) a first container comprising a priming composition comprising a recombinant measles virus; and
b) a second container comprising a boosting composition comprising a recombinant adenovirus,
wherein the recombinant measles virus and the recombinant adenovirus each comprise a transgene that encodes an epitope of a foreign antigen.

15. The kit of claim 14, wherein the epitope is from a protein of a bacterium, a virus or a parasite.

16. The kit of claim 15, wherein the epitope is from HIV Gag protein.


SEQUENCE LISTING (4 sequences)

SEQ ID NO: 1 - DNA, 1533 bp, Simian immunodeficiency virus
  CDS (1)..(1533)
atgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaacaggaacaacagaaactatgccaaaaacaagtagaccaacagcaccatctagcggcagaggaggaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgccaatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtag

SEQ ID NO: 2 - protein, 510 aa, Simian immunodeficiency virus
MGVRNSVLSGKKADELEKIRLRPNGKKKYMLKHVVWAANELDRFGLAESLLENKEGCQKILSVLAPLVPTGSENLKSLYNTVCVIWCIHAEEKVKHTEEAKQIVQRHLVVETGTTETMPKTSRPTAPSSGRGGNYPVQQIGGNYVHLPLSPRTLNAWVKLIEEKKFGAEVVPGFQALSEGCTPYDINQMLNCVGDHQAAMQIIRDIINEEAADWDLQHPQPAPQQGQLREPSGSDIAGTTSSVDEQIQWMYRQQNPIPVGNIYRRWIQLGLQKCVRMYNPTNILDVKQGPKEPFQSYVDRFYKSLRAEQTDAAVKNWMTQTLLIQNANPDCKLVLKGLGVNPTLEEMLTACQGVGGPGQKARLMAEALKEALAPVPIPFAAAQQRGPRKPIKCWNCGKEGHSARQCRAPRRQGCWKCGKMDHVMAKCPDRQAGFLGLGPWGKKPRNFPMAQVHQGLMPTAPPEDPAVDLLKNYMQLGKQQREKQRESREKPYKEVTEDLLHLNSLFGGDQ

SEQ ID NO: 3 - DNA, 1539 bp, Human immunodeficiency virus
  CDS (1)..(1539)
atgggcgccagggccagcgtgctgagcggcggcgagctggacaggtgggagaagatcaggctgaggcccggcggcaagaagaagtataagctgaagcacatcgtgtgggccagcagggagctggagaggttcgccgtgaaccccggcctgctggagaccagcgagggctgcaggcagatcctgggccagctgcagcccagcctgcagaccggcagcgaggagctgaggagcctgtacaacaccgtggccaccctgtactgcgtgcaccagaggatcgagatcaaggacaccaaggaggccctggacaagatcgaggaggagcagaacaagtccaagaagaaggcccagcaggccgccgccgacaccggccacagcagccaggtgagccagaactaccccatcgtgcagaacatccagggccagatggtgcaccaggccatcagccccaggaccctgaacgcctgggtgaaggtggtggaggagaaggccttcagccccgaggtgatccccatgttcagcgccctgagcgagggagccaccccccaggacctgaacaccatgctgaacaccgtgggcggccaccaggccgccatgcagatgctgaaggagaccatcaacgaggaggccgccgagtgggacagggtgcaccccgtgcacgccggccccatcgcccccggccagatgagggagccccgcggcagcgacatcgccggcaccaccagcaccctgcaggagcagatcggctggatgaccaacaacccccccatccccgtgggcgaaatctacaagaggtggatcatcctgggcctgaacaagatcgtgaggatgtacagccccaccagcatcctggatatcaggcagggccccaaagagcccttcagggactacgtggacaggttctacaagaccctgcgcgccgagcaggccagccaggaggtgaagaactggatgaccgagaccctgctggtgcagaacgccaaccccgactgcaagaccatcctgaaggccctgggacccgccgccaccctggaggagatgatgaccgcctgccagggcgtgggcggccccggccacaaggccagggtgctggccgaggccatgagccaggtgaccaacaccgccaccatcatgatgcagaggggcaacttcaggaaccagaggaagatggtgaagtgcttcaactgcggcaaggagggccacaccgccaggaactgccgcgcccccaggaagaagggctgctggaagtgcggcaaggagggccaccagatgaaggactgcaccgagaggcaggccaacttcctgggcaagatctggcccagctacaagggcaggcccggcaacttcctgcagagcaggcccgagcccaccgccccccccttcctgcagagcaggcccgagcccaccgccccccccgaggagagcttcaggagcggcgtggagaccaccacccctcctcagaagcaggagcccatcgacaaggagctgtaccccctgaccagcctgaggagcctgttcggcaacgaccccagcagccagtga

SEQ ID NO: 4 - protein, 512 aa, Human immunodeficiency virus
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSSQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNTATIMMQRGNFRNQRKMVKCFNCGKEGHTARNCRAPRKKGCWKCGKEGHQMKDCTERQANFLGKIWPSYKGRPGNFLQSRPEPTAPPFLQSRPEPTAPPEESFRSGVETTTPPQKQEPIDKELYPLTSLRSLFGNDPSSQ